SEQUENCE LISTING (22 sequences)

SEQ ID NO: 1 - DNA, 10971 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (1)..(10970)
agagcatcttttggggggagggaattcagcggatcagtcttaagaggagcttttttttggagcgagaaatcatataaaataaaatgaaataaaacaaggaggaaggcaaccagctgttaggggaaaaataaggcagataaaggagcggggagagaaattaattgccaaccaggaggagttgggctgtatttttcaaaggtggggagagtggagcacacaccttgaggaggaaagcgagaaagaaaagaaaaaagcaagtggaaaggggggctcgcccaagaagggtgaagaagcgaagaaagtcgaggcgccgaggctcccaaagctggcagctccgggtggcggtgcaggggcgaagggggggcggggggaaccgtcggacatgcggctctggagttgggtgctgcacctggggctgctgagcgccgcgctgggctgcgggctggccgagcgtccccgccgggcccggagagacccgcgggccggccgacccccgcgccccgccgccggcccggccacctgcgccacccgggcggcccgcggccgccgcgcctcgccgccgccgccgccgccgccgggcggtgcctgggaagccgtgcgcgtcccccggcggcggcagcagcgggaggcgaggggcgccaccgaggagccgagcccgccgagccgggcgctctatttcagcgggcgaggcgagcagctgcgcctccgggccgacctcgagctgccccgggacgcgttcacgctgcaagtgtggctgcgagcggaggggggccagaggtctccggcagtgatcacagggctgtatgacaaatgttcttatatctcacgtgaccgaggatgggtcgtgggcattcacaccatcagtgaccaagacaacaaagacccacgctactttttctccttgaagacagaccgagcccggcaagtgaccaccatcaatgcccaccgcagctacctcccaggccagtgggtatacctagctgccacctatgatgggcagttcatgaagctctatgtgaatggtgcccaggtggccacctctggggaacaagtgggtggcatattcagcccactgacccagaagtgcaaagtgctcatgttagggggcagtgccctgaatcacaactaccggggctacatcgagcacttcagtctgtggaaggtggccaggactcagcgggagatactgtctgacatggaaacccatggcgcccacactgctctacctcagctcctcctccaggagaactgggacaatgtgaagcatgcctggtcccccatgaaggatggcagcagccccaaagtggaattcagcaatgcccacggctttctgctggacacgagtctggagcctcctctgtgcggacagacattgtgtgacaacacagaggtcattgccagctacaatcagctctcaagtttccgccagcccaaggtggtgcgctaccgcgtggtcaacctctatgaagatgatcataagaacccgacggtgacgcgcgagcaggtggacttccagcaccatcagctggctgaggccttcaagcaatacaacatctcctgggagctggacgtgctggaggtgagcaactcctcccttcgccgccgcctcatcctggccaactgtgacatcagcaagattggggatgagaactgtgaccccgagtgcaaccacacgctgacgggccacgacggcggggattgccgccacctgcgccaccctgccttcgtgaagaagcagcacaacggggtgtgtgacatggactgcaactatgaacggttcaactttgatggtggagagtgctgtgaccctgaaatcaccaatgtcactcagacttgctttgaccccgactctccacacagagcctacttggatgttaatgagctgaagaacattcttaaattggatggatcaacacatctcaatattttctttgcaaaatcctcagaggaggagttggcaggagtagcaacttggccatgggacaaggaggccctgatgcacttaggtggcattgtcttgaacccatctttctatggcatgcctgggcacacccacaccatgatccatgagattggtcacagcctgggcctctatcacgtcttccgaggcatctcagaaatccagtcctgcagtgacccctgcatggagacagagccctccttcgagactggagacctctgcaatgataccaacccagcccctaaacacaagtcctgtggtgacccagggccaggaaatgacacctgtggctttcatagcttcttcaacactccttacaacaacttcatgagctatgcagatgacgactgtacggactccttcacgcccaatcaagtcgccagaatgcactgttacctggacctggtctaccagggctggcagccctccaggaaaccagcgcctgttgccctcgccccccaagttctgggccacacaacggactctgtgacactggagtggttcccacctatagatggccatttctttgaaagagaattgggatcagcatgtcatctttgcctggaagggagaatcctggtgcagtatgcttccaacgcttcctccccaatgccctgcagcccatcaggacactggagccctcgtgaagcagaaggtcatcctgatgttgaacagccctgtaagtccagtgtccgcacctggagcccaaattcagctgtcaacccacacacggttcctccagcctgccctgagcctcaaggctgctacctcgagctggagttcctctaccccttggtccctgagtctctgaccatttgggtgacctttgtctccactgactgggactctagtggagctgtcaatgacatcaaactgttggctgtcagtgggaagaacatctccctgggtcctcagaatgtcttctgtgatgtcccactgaccatcagactctgggacgtgggcgaggaggtgtatggcatccaaatctacacgctggatgagcacctggagatcgatgctgccatgttgacctccactgcagacaccccactctgtctacagtgtaagcccctgaagtataaggtggtccgggaccctcctctccagatggatgtggcctccatcctacatctcaataggaaattcgtagacatggatctaaatcttggcagtgtgtaccagtattgggtcataactatttcaggaactgaagagagtgagccatcacctgctgtcacatacatccatggaagtgggtactgtggcgatggcattatacaaaaagaccaaggtgaacaatgcgacgacatgaataagatcaatggtgatggctgctcccttttctgccgacaagaagtctccttcaattgtattgatgaacccagccggtgctatttccatgatggtgatggggtatgtgaggagtttgaacaaaaaaccagcattaaggactgtggtgtctacacgccccagggattcctggatcagtgggcatccaatgcttcagtatctcatcaagaccagcaatgcccaggctgggtcatcatcggacagccagcagcatcccaggtgtgtcgaaccaaggtgatagatctcagtgaaggcatttcccagcatgcctggtacccttgcaccatcagctacccatattcccagctggctcagaccactttttggctccgggcgtatttttctcaaccaatggttgccgcagctgtcattgtccacctggtgacggatgggacatattatggggaccaaaagcaggagaccatcagcgtgcagctgcttgataccaaagatcagagccacgatctaggcctccatgtcctgagctgcaggaacaatcccctgattatccctgtggtccatgacctcagccagcccttctaccacagccaggcggtacgtgtgagcttcagttcgcccctggtcgccatctcgggggtggccctccgttccttcgacaactttgaccccgtcaccctgagcagctgccagagaggggagacctacagccctgccgagcagagctgcgtgcacttcgcatgtgagaaaactgactgtccagagctggctgtggagaatgcttctctcaattgctccagcagcgaccgctaccacggtgcccagtgtactgtgagctgccggacaggctacgtgctccagatacggcgggatgatgagctgatcaagagccagacgggacccagcgtcacagtgacctgtacagagggcaagtggaataagcaggtggcctgtgagccagtcgactgcagcatcccagatcaccatcaagtctatgctgcctccttctcctgccctgagggcaccacctttggcagtcaatgttccttccagtgccgtcaccctgcacaattgaaaggcaacaacagcctcctgacctgcatggaggatgggctgtggtccttcccagaggccctgtgtgagctcatgtgcctcgctccaccccctgtgcccaatgcagacctccagaccgcccggtgccgagagaataagcacaaggtgggctccttctgcaaatacaaatgcaagcctggataccatgtgcctggatcctctcggaagtcaaagaaacgggccttcaagactcagtgtacccaggatggcagctggcaggagggagcttgtgttcctgtgacctgtgacccacctccaccaaaattccatgggctctaccagtgtactaatggcttccagttcaacagtgagtgtaggatcaagtgtgaagacagtgatgcctcccagggacttgggagcaatgtcattcattgccggaaagatggcacctggaacggctccttccatgtctgccaggagatgcaaggccagtgctcggttccaaacgagctcaacagcaacctcaaactgcagtgccctgatggctatgccatagggtcggagtgtgccacctcgtgcctggaccacaacagcgagtccatcatcctgccaatgaacgtgaccgtgcgtgacatcccccactggctgaaccccacacgggtagagagagttgtctgcactgctggtctcaagtggtatcctcaccctgctctgattcactgtgtcaaaggctgtgagcccttcatgggagacaattattgtgatgccatcaacaaccgagccttttgcaactatgacggtggggattgctgcacctccacagtgaagaccaaaaaggtcaccccattccctatgtcctgtgatctacaaggtgactgtgcttgtcgggacccccaggcccaagaacacagccggaaagacctccggggatacagccatggctaaggaaggacaagaagttgtcaaagaattcccaacgccaggacccacatccctttggtattgatttcacagtcagctgctcaacggaatggcctctccacaccagggatccttagcacccaaccggtctgcctttaattttacccaggaaggactcacattggggcgaatgaaccaagtttcgccatgctggatgatgaaatggattcccatcccaaagtctgagatggattgcatatacagtgtgcagtcccagagcctcctaaaattctagccatttgtcacacaaccacagcaagaaacgtgttctatatctagagtgtgcccatctgtgtttagtacacatgcatgcatacacacccatacaaacatctgtgtgagggcagttctggagatgagcagagagagaccggaataaactcaatcttttctttcccaagctcctagccaacactatccttgggagaaagaaatttgcagaaactgctaagaccaagtgtggagatgtcaagctagttcacactctgaggctcagaatatgtaggacatgcacaattgtgcagtcctttgggattggaagtgaaacagtctgtgatcccctaccttctagggaactaggacctaggaagaggtaaagattatcaggtatgcaaagcgccccaattcttctgctgccatgggggattttaccccaactccagggttcgaggccaatctgagaatggcttaggattgcaatgtcaaggtattatatcagccccttgcttgaggcttgaggtcataatatccctctaggacttacctgttcccccagatcttgccttgggaccacatttgctgctacttttcctgctgctctatcctatacattgaataatccaagatggtagaactaggttaggaaaaattccacacaaccaaacagtctgccttaaaagtgacccacatttttccatagctcctcactttttagcccttctgcaagagaaaaaccctcatgggtccacatggtgagaagttaagtttcctgtaagtgggcctctcaccctggaaaggagttgagggacatcagatgctggaaccctcactgaaagtccagaatgtctaagccagtgttagattttgtaaacaagtggaacagtgttaaatttctatgatgttggagccatccagagactactggaattgtcgagacttttggattattatccttatccttatcctaatcttcctagcccttcaggctagagtaggctttgatcctgagaaccttgctgttgctctgaggagatataattctgggagaaagaatcttttataagaacagtacagattgttctcaagagggccatcagaaggaagccaaagagttcacagcctcagcaccaacaactcaacatggtcatcatgttttctatatggtttttccagctagcagtactcccttccatacctgtgactgggcagtgcttttctctctcccatgtctagcctccaaaagttaagtgaaaattagtcaactgcacgtggaagcccccaccactttggggatctctttatttcttttcagccagggacctgtccactccctttgaattaatatgggaagaaattaatacaggatgaactggagagaagggttgagtgtggcatactttctgaaacctggagctgggaattgcggagaagggaaggtctagactagttacatcacatagggattactgtaaatcaagtcatctcaagtctagtgaagacagccaacagaaacaaaacctagcatagggatagaaaataccatgcacgtgtgcagccccacctaattcctgcatccaaggcaggtgttgttaatctatcatagcacttaaaaaaaaaaaaaaaaagagaccaaaaataactttaggaaccaccatattatatcactcccaatagcactgacctggtgatcaaaaacacttgagaagacatctattggccatctctggccaattacactaagaaacatatcaaggtgcttttggcacaggtgcccacaaatacggatgcagtgctgagatagtttatgagacttgtaccatttcacaaactctgaaattgggttccatattggcaaggctgccacagttgttaagaataatcctctatgtttcttcctcacaaaaccatatctcatttatatccagaccattacttcactataattacaaggacaaattattagcaagaaataagaatagtattagaagaattgatcctattttgaacccctctccagtatcttcacactcttgtcaactctccaggcctctctcttgccctgagttatcagcctgtgtggtgttaactaccttagaaggtacaagctaagaaatgtaacagtatcaaccctcccagttgcttaattatacccataggtaatacaaaaagctctgaagacccaaagatgacattactaatgatgtgatttcaggagccacagaagaaccttaccagcttccctcaaatcagtccttatcctctttctatcttcactcccatcatcatctattttcacactatccagctaagcaaagattcctggaggctgacttgtatcttcagactcacagagtgaattcagctcttctgaatcaagacccacccagtctctttcattcagacctgttgctaacaaatttatatttgccaaggatattaggcaaaagaggctacttgattggtggccaacctcgtgcccacatggaaggtatctttaatagggtcttttcaaaccttagtggaggagggtcagctcaatttgggcaatgcatttgttcccagtttcattttcttcctgggaattaactcgtcatttcattccttcagtcatcttctgtgtaggtgaccggagcactgagaggcagctctgatgcactattgtgtgtcagcagctcaaaggccctaaaacactgaaggttctgcatctgaagtattagattgttagcagcaaaatatgaaagatgaggtggacagtcctctaagccctatttagggaagcttttccaagccacaatcttaactacctacccaaaggatttgcattacccccagattctgtgccaacaaccttttaaggaaatacagtccttgggaaatgagttttgatggtgaattggggtgttaaggaagggaaagattgtcatagatggtagggctttgaaaatgcagggtatcagctgccactcctggcttcaacacattgagtcactgcctagacggttctcttggtcttattcccatcctggccaatgcttaaatactatttgttgaaaataattctttgagacagatttcagctacctcccttccaggttcgatttaacttggttgtaattgtcaatttgttgttataggtcttacctgtgtgaaagaaagaaaaagaaagaaagaaagaaagagaaaggaaattataaggtcaagttaacagttttgaggttttgtgtttttttctggaactacttcaagtgagaaaataaaaaaaaatggtgacaaagctgtacagatagagataatagaagacaaagagattaaaaggaaataaaaatgcatgattaaaaactaagaataaaaaacctatttttatgtttcctaaaggaaattgtttattctacagcctcagtaggtagacacaaacataaagatttccctagaagacatagagtgggatttgataacactgtctgttattttctgtacattgtggtaggtccaggaaatatgacattttcccccttgatgtgttattgttgttgttgggtggggtgggcattttgtttatttgtttggtggcaatcagtggtagtagggagtgggagggcttatattggtttttccagctattaaggggacatattgtgtcgttgtgcttttcacgttataaaatgtttatatttaccagtacagcactgggctttataaagactgcactcagaaccacactgcacagtccagttttttaaaaagctgctacatgacagacaggtaatcccactgagtgagttttgagaaacaaatcaaacgaagtaaacaagaaacataaaaaccaaatagcaaatgaataaaagcctgttcttgtaacttattcaacttttgccaaattcctaccaatcacttgctttttaaaagaaatgtataatagccaaaagagaaattatgtccctgttgtacagaagttagaatttttgactccaggcagcagtttgctcagtgatcttgaacaagttatccaattgcctctacatttgcatcagtttctctagctgcaaaatggggataatactatatacctacctcacagtgggagggcaggagattttgaggccctgaggttttaggtgggctgtgagggccaacgcttgacacaaagtccatgggttattattcaagaatgcacaggcccatcggccttttagaaagacaagacagggagtgcttgtttgatatttcaaggaataaagccggagctcctgaattgtagtccaccttaaaagagagacctgtattggagaatattttatttttttggcaaatttgatcttaccctttaccagttctataatttggttaaaagctgattatgtcctacaatgtcaaagtcagctaactgtcgtctacttaagacttctggtcatttccaacttatagaggaagggagtctctaaaatctcttcttcagaaggcacctcacttctcagacttaaaattccacatcaagtgttccattaaaagaagataaggcattctgagtgcaaacaaatgggggcttcttaaactacacaccagcagtcagtgaggaaaactttgaacaattattgagttgctttcttgggtctctataatcaataacctgtctgcagatatctatctatataaagatattatatataaatataaatttacatatatatgcacatgtatatatagttgtacatatatgtgtgtatatatatacttaaatgtaatatttacaaaataaaactgtgatctcgtctagagaaaatgtattcatattacaaactgctcttccatatttatgtaccatattatacctttttattattgttataattattatgggtatttctaattaatatgatgttgaaacctgtttggcaccttctggaagctaccaaaaaaatgacactccattgaagtgcttaaaagctgttctcataagaattcttctggcctattgtaaaaaagaaaaaaaaaaagaaaaagaagaaagacacaaagaaaataatctaaacaccaaaaactaaacacaattccaatcctttttctgtacctcacgcgcataaatttgctgctcctatttttttttctgtttatgtgtttttatggatctaagttaaatcttttggcaatatataaaaatgtaaatagtaaactttatttattaagaatgtcatcttttttaatttatatttacacaattgttcatctaatttattttttctatacagttttaaatactcagacatattttgctgttcatgatatttttatcctgttctcatggatttgttttcccatactgttttctctgatctcaattacaggttggatctcacaaataataatgtcagagacagaaatattttgccactgttgattactatactttaaagttctatattatgaaaatatataatagcttgtacgcttca

SEQ ID NO: 2 - DNA, 25 bp, Homo Sapiens
  ref <308>: DATABASE ACCESSION NUMBER: genbank/NM_002581.4
  ref <309>: DATABASE ENTRY DATE: 2018-11-23
  ref <313>: RELEVANT RESIDUES IN SEQ ID NO: (9725)..(9749)
cagggagtgcttgtttgatatttca

SEQ ID NO: 3 - DNA, 25 bp, Homo sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (5823)..(5847)
gcagtcctttgggattggaagtgaa

SEQ ID NO: 4 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (4914)..(4938)
gcgagtccatcatcctgccaatgaa

SEQ ID NO: 5 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (4661)..(4685)
cagtgtactaatggcttccagttca

SEQ ID NO: 6 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (3736)..(3760)
cagcgtgcagctgcttgataccaaa

SEQ ID NO: 7 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (3308)..(3332)
cgacaagaagtctccttcaattgta

SEQ ID NO: 8 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (3305)..(3329)
tgccgacaagaagtctccttcaatt

SEQ ID NO: 9 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (1854)..(1878)
cacacagagcctacttggatgttaa

SEQ ID NO: 10 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (1077)..(1101)
cccagaagtgcaaagtgctcatgtt

SEQ ID NO: 11 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (2449)..(2473)
gacactggagtggttcccacctata

SEQ ID NO: 12 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (2491)..(2515)
aagagaattgggatcagcatgtcat

SEQ ID NO: 13 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (4256)..(4280)
caagtctatgctgcctccttctcct

SEQ ID NO: 14 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (5796)..(5820)
cagaatatgtaggacatgcacaatt

SEQ ID NO: 15 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (5835)..(5859)
gattggaagtgaaacagtctgtgat

SEQ ID NO: 16 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (6075)..(6099)
cagatcttgccttgggaccacattt

SEQ ID NO: 17 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (6426)..(6450)
gagccatccagagactactggaatt

SEQ ID NO: 18 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (6644)..(6668)
cagcaccaacaactcaacatggtca

SEQ ID NO: 19 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (9495)..(9519)
cagcagtttgctcagtgatcttgaa

SEQ ID NO: 20 - DNA, 20 bp, Homo Sapiens
  ref <308>: DATABASE ACCESSION NUMBER: uswestensembl.org/ENSG00000182752
  ref <309>: DATABASE ENTRY DATE: 2020-02-01
  ref <313>: RELEVANT RESIDUES IN SEQ ID NO: (1)..(20)
ccctattgcatgtccattaa

SEQ ID NO: 21 - DNA, 21 bp, Homo Sapiens
  ref <308>: DATABASE ACCESSION NUMBER: uswestensembl.org/ENSG00000182752
  ref <309>: DATABASE ENTRY DATE: 2020-02-01
  ref <313>: RELEVANT RESIDUES IN SEQ ID NO: (1)..(20)
tgtcctgggacgtcttaaagt

SEQ ID NO: 22 - DNA, 25 bp, Homo Sapiens
  ref <308>: DATABASE ACCESSION NUMBER: genbank/NM_002581.5
  ref <309>: DATABASE ENTRY DATE: 2018-11-23
  ref <313>: RELEVANT RESIDUES IN SEQ ID NO: (9765)..(9789)
gctcctgaattgtagtccaccttaa

(19) United States
(12) Patent Application Publication
(10) Pub. No.: US 2021/0380986 A1
(43) Pub. Date: Dec. 9, 2021

(54) RNA DRUGS AND METHOD OF TREATING AGE-RELATED DISEASE

(71) Applicant: Richelle Gayle Cutler, Oro Valley, AZ (US)

(72) Inventor: Richelle Gayle Cutler, Oro Valley, AZ (US)

(21) Appl. No.: 17/306,908

(22) Filed: Jun. 2, 2021

Related U.S. Application Data
(60) Provisional application No. 63/019,305, filed on May 2, 2020.

(51) Int. Cl. C12N 15/113

(52) U.S. Cl. CPC C12N 15/1137; C12N 2310/315; C12N 2310/11; C12N 2310/3513; C12N 2310/3231; C12N 2310/322; C12N 2310/3515; C12N 2310/321

(57) ABSTRACT

Mouse models show that decreasing the expression of pregnancy-associated plasma protein-A (PAPP-A) can reduce age-related muscle atrophy, cancer, atherosclerosis, and increase immune function and longevity. The invention describes RNA agents designed to reduce the expression of pregnancy-associated plasma protein-A. The invention further describes delivering these RNA agents to subjects in vivo as a method of preventing age-related disease and thymic atrophy.

### BACKGROUND

Decreased immunity in the elderly is responsible for their high mortality rate from COVID-19, as well as other infectious diseases and cancer. Vaccines for preventing COVID-19 are being developed at a furious pace. Unfortunately, these vaccines are unlikely to adequately protect the most susceptible population, the elderly. Indeed, a coronavirus vaccine mouse model showed limited protection in aged mice (Deming et al. 2006).

An optimal vaccine response should include both a prolonged humoral response and a type 1 cytotoxic T-cell response to eliminate cells harboring the virus. However, the cytotoxic T cell response is significantly impaired in the elderly because of decreased naïve T cell numbers (Weinberger et al. 2008). Interestingly, mice without pregnancy-associated plasma protein-A (PAPP-A) gene expression maintain their naïve T cells numbers with age and a robust immune response.

PAPP-A is a novel zinc metalloproteinase that is secreted and binds to the cell surface. At the cell surface, PAPP-A cleaves extracellular insulin growth factor receptor binding proteins 4 and 5 to release insulin growth factor 1 (IGF1). Reduction of PAPP-A activity reduces the local availability of IGF1. The PAPP-A knock-out and adult conditional knock-out mice both have extended lifespans compared to wildtype mice of approximately 40% and 20%, respectively. Inactivating PAPP-A decreases inflammation, atherosclerosis, muscle atrophy, and cancer in old mice. Furthermore, mice treated with antibodies to deactivate PAPP-A have approximately 70% reduction in atherosclerotic plaque. Thus, reducing PAPP-A activity in humans could be therapeutic. Unfortunately, researchers have yet to identify a pharmacological agent that blocks PAPP-A activity.

### SUMMARY

Animal models show that decreasing local IGF-1 by blocking PAPP-A activity can maintain naïve T-cell numbers and reduce age-related disease. The present invention describes new RNA agents to reduce PAPP-A expression and a method of preventing age-related disease by delivering said RNA agents to subjects in vivo. The RNA agents are delivered in microvesicles or attached to peptide or lipid conjugates, which can be targeted to PAPP-A expressing cells.

### DETAILED DESCRIPTION

Decreasing PAPP-A expression in humans could have therapeutic potential. IGF does not have regulatory feedback on PAPP-A expression, so blocking PAPP-A expression should translate into reduced activity. The invention herein describes new antisense oligonucleotides (ASO) to reduce PAPP-A expression (SEQ ID NO. 2-22).

RNA agents with the largest PAPP-A knockdown in cultured fibroblasts, kidney mesangial cells, adipocytes, and osteoblast, were selected for testing in mice. PAPP-A knockdown was measured by quantitative RT-PCR and PAPP-A immunoassay. Appropriate controls and assays ensured that the RNA agents had no off-target effects or immune stimulation.

RNA modifications can increase target affinity, RNase resistance, duration, and minimize immune stimulation.

The RNA chemical modifications may include a combination of the following: 2′-O-methyl modification alternating with 2′-fluorine at CA, UA, and UG, 2′-O-methoxyethyl on the flank and central antisense nucleotides, ribose analog cyclohexenic, glycolic nucleic acid at the 5′ antisense strand, and locked nucleic acid (LNA) medication at the 3′ antisense end, 2′ deoxythymidine substitution of uridine, replacing the phosphodiester bond with an amide bond, 5′ methylphosphonate or 5′ (E)-vinylphosphonate at the end of the antisense strand, replace oxygen of phosphate with phosphorothioate at 3′ nucleotide. The ASO modifications may further include methylene bridge LNA, 2′-O-methyl, 2′-O-methoxyethyl, 2′-fluorine, phosphorothioate linkages, ASO gapmers with terminal nucleic acid modification, ASO 2′-fluoroarabinonucleic acid, and amide linked nucleotides.

Another aspect of the invention is delivering said RNA agents to human subjects in vivo as a method of preventing age-related disease. The RNA agents are delivered in microvesicles, such as exosomes or as lipid/peptide-conjugates. In one embodiment, clinical-grade exosomes are produced in large batches from an immortalize mesenchymal cell line. Mesenchyme cells do not express major histocompatibility complex (MHC) proteins and are therefore less immunogenic. RNA agents are loaded directly into the exosome production cell by transfection or by direct liposome fusion. Alternatively, RNA agents are loaded post-generation by electroporation. Exosomes are isolated from culture supernatant by size exclusion chromatography for optimal scale, cost, and purity. For quality control, exosome RNA is quantified by RNA isolation, reverse transcription, and quantitative RT-PCR.

Purified exosomes are lyophilized in trehalose, encapsulated, and stored at room temperature (Charoenviriyakul et al. 2018). Milk exosomes administered orally to adult mice penetrates their intestinal mucosal barrier and disseminates throughout the body. Thus, it is possible that therapeutic exosomes formulated in capsules can reach target cells with oral dosing in humans.

PAPP-A is expressed in visceral adipose tissue (100), kidneys (200), lungs, colon, bladder, female organs, heart, adrenal and thyroid gland, etc. (FIGS. 1A and 1B). Visceral fat has the highest expression of PAPP-A, and PAPP-A expression in kidneys increases 2-fold with age. Exosomes can be targeted to PAPP-A expressing cells. This is accomplished by targeting the cell-surface receptors in PAPP-A expressing cells. Receptors can be targeted with the single-chain antigen-binding fragment (Fab) of antibodies or a peptide ligand. Cell-surface receptors matching PAPP-A tissue-specific expression were identified using the human protein atlas. For instance, the tissue expression of natriuretic peptide receptor 1 (NPR1) overlaps that of PAPP-A (FIGS. 3A and 3B). However, NPR1 is not expressed in bone, whereas PAPP-A is expressed in osteoblasts. This incongruity works though since decreasing PAPP-A in osteoblasts could reduce bone density. Other possible receptors for targets are DC-SIGN (DC-209), insulin growth factor 1 receptor (IGF1R), and platelet-derived growth factor receptor B (PDGFRB).

The natriuretic peptide receptor ligand is an atrial natriuretic peptide (ANP). ANP can be expressed on the exosome surface by fusing the DNA of exosome cell membrane proteins, such as Lamp2b, with DNA encoding ANP, and cloning the fused DNA into an exosome production cell line. The exosome ANP-fusion protein will then bind NPR1 on PAPP-A expressing cells. NPR1 undergoes internalization and endosomal trafficking to lysosomes. A significant hurdle for delivery is the degradation of endosome contents in lysosomes. However, exosomes are capable of fusing with the endosome/lysosome membrane in a pH-dependent manner, releasing their contents into the cytoplasm. Exosomes can penetrate the blood-brain barrier and can therefore enter the brain to reduce PAPP-A expression in the brain.

In another embodiment, RNA agents are delivered conjugated to a lipid or peptide. RNA conjugated to a lipid such as eicosapentaenoic (EPA), cholesterol, or docosahexaenoic acid (DHA) can aid in tissue distribution. A combination of different lipid conjugates can increase desired tissue distribution. Attaching peptides to the RNA can target delivery. For instance, the peptide CSKC, which mimics IGF1, targets delivery to IGFR1 expressing cells. RNA conjugates can be administered by subdermal injection. With optimal delivery and chemical modifications, PAPP-A downregulation can last 6 months.

### BRIEF DESCRIPTION OF THE DRAWINGS

FIG. 1A depicts the expression pattern of PAPP-A in women (left) as displayed in the human protein atlas (Uhlén et al. 2015). Reference NO. 100 points to visceral fat and reference NO. 200 points to a kidney.

FIG. 1B depicts the expression pattern of PAPP-A in men (right) as displayed in the human protein atlas (Uhlén et al. 2015). Reference NO. 100 points to visceral fat and reference NO. 200 points to a kidney.

FIG. 2 shows the mean PAPP-A mRNA transcript expression level in different tissues from men and women, wherein pTPM designates protein-coding transcripts per million.

FIG. 3A depicts the expression pattern of NPR1 in women (left) as displayed in the human protein atlas (Uhlén et al. 2015). Reference NO. 100 points to visceral fat and reference NO. 200 points to a kidney.

FIG. 3B depicts the expression pattern of NPR1 in men (right) as displayed in the human protein atlas (Uhlén et al. 2015). Reference NO. 100 points to visceral fat and reference NO. 200 points to a kidney.

FIG. 4 shows the mean NPR1 mRNA transcript expression level in different tissues from men and women, wherein pTPM designates protein-coding transcripts per million.

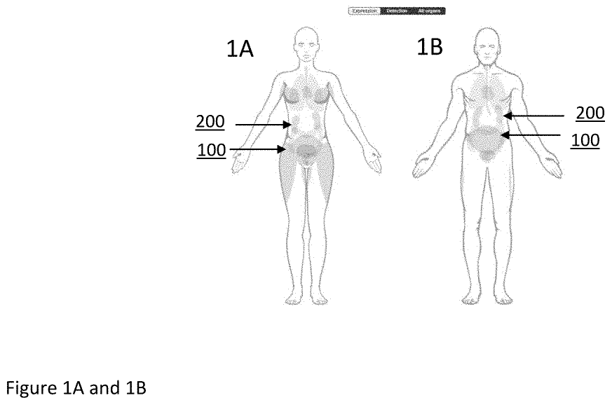

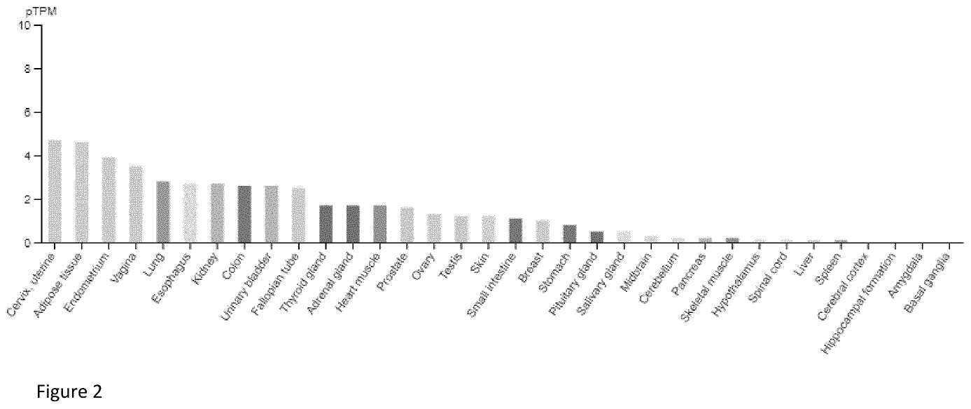

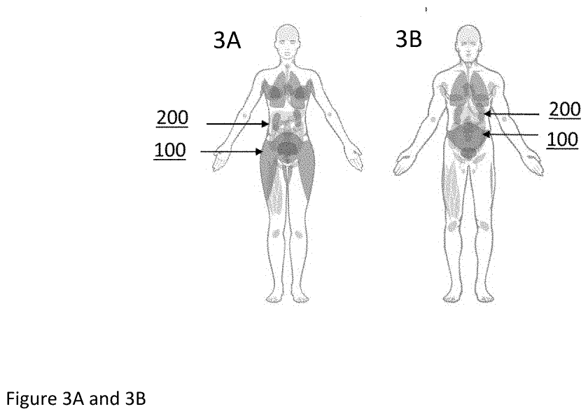

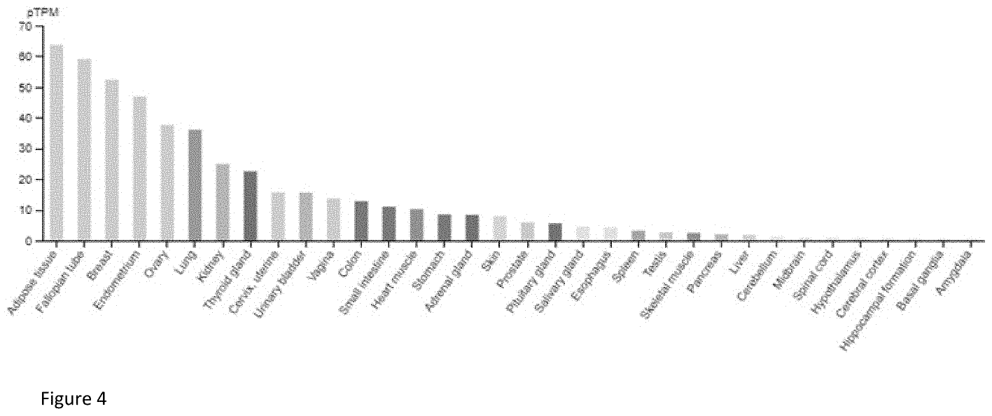

### CONCLUSION

The invention describes new RNA agents that block the expression of PAPP-A. The invention further describes a method of preventing age-related diseases by delivering the said RNA agents to subjects in vivo. Blocking PAPP-A expression by the RNA agents will maintain immunity, and decrease inflammation, atherosclerosis, and cancer growth. There has been a longstanding need for this type of therapeutic.

### SEQUENCES

The sequences are disclosed in a 19 Kb text file named 10002B-US-NP_Sequences-as-filed.txt, created on May 3, 2021.

1-20. (canceled)

21. An antisense oligonucleotide of 10-25 nucleotides in length, which comprises a contiguous sequence of at least 10 nucleotides that are 90% complementary to the pregnancy-associated plasma protein A mRNA (SEQ ID NO 1) targeted sequences listed in SEQ ID NO 2-22.

22. The antisense oligonucleotide of claim 21, wherein one or more oligonucleotide modifications are selected from phosphorothioate linkages, 2′-O-methyl groups, 2′-O-methoxyethyl, locked nucleic acid with methylene bridge, 2′-fluorine, amide linkage, 2′-fluoroarabinonucleic acid, 2′deoxythymidine substitution of uridine, glycolic nucleic acid at the 5′ end antisense strand, and the ribose analog cyclohexenic.

23. A method of preventing an age-related disease, wherein antisense oligonucleotide of 10-25 nucleotides in length, which comprises a contiguous sequence of at least 10 nucleotides and 90% complementary to the pregnancy-associated plasma protein A mRNA (SEQ ID NO 1) targeted sequences listed in SEQ ID NO 2-22.

24. A method according to claim 23, wherein one or more oligonucleotide modifications are selected from phosphorothioate linkages, 2′-O-methyl groups, 2′-O-methoxyethyl, locked nucleic acid with methylene bridge, 2′-fluorine, amide linkage, 2′-fluoroarabinonucleic acid, 2′deoxythymidine substitution of uridine, glycolic nucleic acid at the 5′ end antisense strand, and ribose analog cyclohexenic.

25. A method according to claim 24, wherein the antisense oligonucleotides are delivered in vivo within microvesicles, such as exosomes.

26. A method according to claim 25, wherein the exosomes are delivered by oral administration.

27. A method according to claim 25, wherein microvesicles target PAPP-A expressing cells with a single chain antigen binding fragment (Fab) or peptide.

28. A method according to claim 27, wherein the Fab targets NPR1.

29. A method according to claim 27, wherein the peptide is selected from ANP-peptide and CSKC-peptide.

30. A method according to claim 24, wherein the antisense oligonucleotide is conjugated to a molecule selected from a lipid, Fab, and peptide.

31. A method according to claim 30, wherein the Fab targets NPR1.

32. A method according to claim 31, wherein the peptide is selected from ANP-peptide and CSKC-peptide.

33. A method according to claim 30, wherein the lipid is comprised of eicosapentaenoic, cholesterol, and docosahexaenoic acid.
